(19) United States
(12) Patent Application Publication
(10) Pub. No.: US 2006/0009372 A1
(43) Pub. Date: Jan. 12, 2006

(54) Low-sulphur odorants for liquid gas

(75) Inventors: Gerd Mansfeld, ESCHERSHAUSEN (DE); Dirk Muller, Dassel (DE); Nadine Tacke, Negenborn (DE)

Correspondence Address: ROYLANCE, ABRAMS, BERDO & GOODMAN, L.L.P., 1300 19TH STREET, N.W., SUITE 600, WASHINGTON,, DC, 20036, US

(73) Assignee: SYMRISE GMBH & CO. KG, HOLZMINDEN (DE)

(21) Appl. No.: 10/525,794

(22) PCT Filed: Aug. 22, 2003
(86) PCT No.: PCT/EP03/09325

§ 371 Date: Feb. 25, 2005

(30) Foreign Application Priority Data
Aug. 27, 2002 (DE) 102 40 028.8

(51) Int. Cl. A61K 8/18

(52) U.S. Cl. 512/8

(57) ABSTRACT

The present invention relates to acrylic acid alkyl ester mixtures containing a low proportion of sulphurous compounds, the use of these to odorize liquid gas, a method for odorizing liquid gas and liquid gas containing these mixtures.

The present invention relates to acrylic acid alkyl ester mixtures containing a low proportion of sulphurous compounds, the use of these to odorize liquid gas, a method for odorizing liquid gas and liquid gas containing these mixtures.

Liquid gas is used in numerous areas. Examples include use as a fuel gas (such as camping gas, motor fuel and automobile fuel, in gas lighters, in the home and in industry), as a propellant (e.g. in sprays) and as a chemical crude gas.

Gas odorization means the addition of strong-smelling substances (odorizers) that act as warning or alarm substances to gases that do not have an inherent small, i.e. to gases that are otherwise essentially or completely odourless.

Liquid gas generally means gases that can be converted at low pressure and at 20° C. into the liquid state. In the strictest sense liquid gas (LPG, or liquefied petroleum gas) essentially comprises propane, propylene, butane and butylene. In the strictest sense liquid gas means propane and n-butane or mixtures of these, which may also contain proportions of unsaturated and/or branched hydrocarbons such as propylene, isobutane, 1-butylene, cis-2-butylene, trans-2-butylene or isobutylene. The requirements on the content of liquid gas are described in DIN 51622 and DVGW Worksheet G 280 (DVGW=Deutscher Verein des Gas- und Wasserfaches e.V—German Gas and Water Association).

Because of its high degree of purity the liquid gas used today is in itself virtually odourless.

If leaks are not detected in good time, explosive gas/air mixtures with a high hazard potential can quickly build up.

For safety reasons, therefore, liquid gas is odorized by the addition of strong-smelling substances. The odorizers are noticeable even at high dilutions and because of their exceptionally unpleasant smell as desired are interpreted by people as an alarm. The odorizer must not only be unpleasant and unmistakable in odour, it must above all represent a warning smell. Therefore the smell of the odorized gas must not be a familiar day-to-day smell in, for example, the kitchen or home.

The normal odorizers for liquid gas usually comprise individual substances or mixtures of individual substances from the group of mercaptans or sulphides such as ethyl mercaptan, n-propyl mercaptan, isopropyl mercaptan, tert.-butyl mercaptan (TBM), tetrahydrothiopene, dimethyl sulphide and diethyl sulphide. Of these, ethyl mercaptan is the most frequently used odorizer for liquid gas.

Mercaptans are extremely well-suited for use as a reliable odorizer of liquid gas. However, within the context of greater care for the environment, it has to be taken into account when such odorized gases are burned sulphur oxides are produced. Several hundred tonnes of these combustion products result across the country every year. Other known problems with mercaptans or sulphides are corrosion, interactions with metal surfaces (e.g. adsorption on container walls) or the incompatibility of metal catalysts with these compounds.

Since a reduction in sulphurous compounds is being sought, tests have now been performed on the development of low-sulphur odorizers.

JP-A 55-104393 describes how odorizers containing alkine and at least 2 compounds selected from a group comprising methyl acrylate, ethyl acrylate, methyl methacrylate, allyl methacrylate, ethyl propionate, methyl-n-butyrate, methyl-iso-butyrate and prenyl acrylate, and as necessary tert. butyl mercaptan are suitable for the odorization of fuel gases. The quantity of odorizer is 50 ppm (mg/kg gas), preferably equal to or greater than 100 ppm with reference to the weight. The best results for LPG were obtained with mixtures containing TBM. Through the addition of 2-butine (50 ppm) to a mixture of methyl acrylate ( 50 ppm), allyl acrylate (100 ppm) and TBM (5 ppm) a better odorizing effect was achieved. The best result was with a mixture of 2-butine (50 ppm), allyl methacrylate (20 ppm), methyl acrylate (20 ppm), methyl-n-butyrate (20 ppm), methyl-iso-butyrate (20 ppm), ethyl propionate (20 ppm) and TBM (5 ppm).

JP-B 51-034841 describes how ethyl acrylate or n-valerianic acid used on their own do not have sufficient odorizing effect because of their olfactory characteristics. The optimum mixture comprised 50-90% by weight of ethyl acrylate, 10-50% by weight n-valerianic acid and optionally triethyl amine. A mixture comprising 60% by weight ethyl acrylate and 20% by weight each of n-valerianic acid and triethyl amine was added to a gaseous fuel gas at the rate of 10 mg/m³.

Odorizers for fuel gases comprising ethyl acrylate (70% by weight) and tert. butyl mercaptan (30% by weight) are known from JP-B 51-021402. This mixture was added to a gaseous fuel gas in a quantity of 5 mg/m³.

Odorous substances for odorizing heating gases comprising a) 30-70% by weight C₁-C₄-alkyl mercaptans, b) 10-30% n-valeraldehyde and/or isovaleralderhyde, n-butyric acid and or isobutyric acid and if necessary c) up to 60% by weight tetrahydrothiopene are described in DE-A 31 51 215. These odorizers were added to heating gas in quantities of 5-40 mg/m³.

Mixtures containing a) 1 part by weight dimethyl sulphide, b) 0.8-3 parts by weight tert. butyl mercaptan and c) 0.1-0.2 parts by weight tert. heptyl mercaptan or 0.05 -0.3 parts by weight tert. hexyl mercaptan for odorizing fuel gases are known from JP-A 61-223094. These mixtures have the smell of tert. butyl mercaptan which is associated with the smell of town gas.

The use of norbomene derivatives for fuel gas odorization is known from JP-A 55056190. 40 mg/kg of a mixture of equal proportions of 5-ethylidene-2-norbornene and 5-vinyl-2-norbornene or 50 mg/kg with a mixture of 80% by weight 5-ethylidene-2-norbornene and 20% by weight ethyl acrylate were added to LPG.

Mixtures for the odorization of town gas containing norbornene or a norbornene derivative and a diluent are described in DE-A 100 58 805.

Mixtures of C₄-C₇-aldehydes and sulphur compounds are described as odorizers in JP-A 50-126004. The odorization of 1 kg of propane was performed with 50 mg of a mixture of 60% by weight valeraldehyde and 40% by weight n-butyl mercaptan. Here the valeraldehyde increases the smell of the n-butyl mercaptan. 2-methyl valeraldehyde was used in a similar manner.

The fact that antioxidants, in particular phenol derivatives are suitable for stabilising mercaptan-containing or alkyl acrylate-containing gas odorizers is known from U.S. Pat. No. 2,430,050 or DE-A 198 37 066.

Alternative low-sulphur odorizers have been sought for the odorization of liquid gas.

The object of the present invention comprises mixtures containing
A) at least two different acrylic acid-C₁-C₆-alkyl esters;
B) at least a compound from the group comprising C₁-C₈-mercaptan, C₄-C₁₂-thiopene, C₂-C₈-sulphide or C₂-C₈-disulphide;
C) at least a compound from the group comprising norbornene, C₁-C₅-carboxylic acids, C₁-C₈-aldehyde, C₆-C₁₄ phenol, C₇-C₁₄ anisole or C₄-C₁₄ pyrazine;
D) if necessary an antioxidant
   for the odorization of liquid gas.

A further object of the present invention is the use of the mixtures in accordance with the invention to odorize liquid gas, a method for odorizing liquid gas and liquid gases containing the mixtures in accordance with the invention.

The acrylic acid-C₁-C₆-alkyl esters are advantageously selected from the group comprising acryl acid methyl esters, acrylic acid ethyl esters, acrylic acid n-propyl esters, acrylic acid iso-propyl esters, acrylic acid n-butyl esters, acrylic acid iso-butyl esters, acrylic acid tert.-butyl esters, acrylic acid n-pentyl esters, acrylic acid iso-pentyl esters and acrylic acid n-hexyl esters.

Preference is for acrylic acid C₁-C₄-alkyl esters, in particular acrylic acid methyl esters, acrylic acid ethyl esters, acrylic acid n-propyl esters, acrylic acid iso-propyl esters, acrylic acid n-butyl esters and acrylic acid iso-butyl esters. Quite particularly preferred acrylic acid C₁-C₄-alkyl esters are acrylic acid methyl esters, acrylic acid ethyl esters and acrylic acid n-butyl esters.

The mixtures in accordance with the invention contain the low-molecular acrylic acid alkyl ester and the high-molecular acrylic acid ester preferably in a weight ratio of 9:1-1:9, more preferably 7:3-3:7, and particularly 3:1-1:3.

The compounds from group A) are contained in the mixtures in accordance with the invention advantageously at 60-97% by weight, preferably 70-95% by weight, with particular preference for 80-95% by weight.

The mercaptans involved can by way of example be ethyl mercaptan, n-propyl mercaptan, isopropyl mercaptan, n-butyl mercaptan, sec.-butyl mercaptan, isobutyl mercaptan, tert.-butyl mercaptan, n-pentyl mercaptan, isopentyl mercaptan, neopentyl mercaptan, n-hexyl mercaptan, isohexyl mercaptan, sec.-hexyl mercaptan, neohexyl mercaptan, tert.-hexyl mercaptan, n-heptyl mercaptan, isoheptyl mercaptan, sec.-heptyl mercaptan, tert.-heptyl mercaptan, n-octyl mercaptan, isooctyl mercaptan, sec.-octyl mercaptan or tert.-octyl mercaptan.

The thiopenes are advantageously thiopenes that are substituted by between 1 to 4, preferably by two, C₁-C₄ alkyl- and/or alkoxy groups. The thiopenes involved can also be hydrated thiopenes, with tetrahydrothiopene being preferred.

The sulphides involved can by way of example be dimethyl sulphide, diethyl sulphide, di-n-propyl sulphide, diisopropyl sulphide, di-n-butyl sulphide, diisobutyl sulphide, ethyl methyl sulphide, methyl-n-propyl sulphide, methyl isopropyl sulphide, methyl isobutyl sulphide, ethyl isopropyl sulphide or isobutylisopropyl sulphide. Preference is for dimethyl sulphide, diethyl sulphide, di-n-propyl sulphide, diisopropyl sulphide, di-n-butyl sulphide and diisobutyl sulphide.

The disulphides involved can by way of example be dimethyl disulphide, diethyl disuphfide, di-n-propyl disulphide, diisopropyl disulphide, di-n-butyl disulphide, diisobutyl disulphide, ethyl methyl disulphide, methyl-n-propyl disulphide, methyl isopropyl disulphide, methyl isobutyl disulphide, ethyl isopropyl disulphide or isobutyl isopropyl disulphide. Preference is for dimethyl disulphide, diethyl disulphide, di-n-propyl disulphide, diisopropyl disulphide, di-n-butyl disulphide and diisobutyl disulphide.

The compounds from group B) are contained in the mixtures in accordance with the invention typically at 1-40% by weight, advantageously 2-30% by weight, and preferably 3-15% by weight.

The norbomenes involved are advantageously those with a molecular weight of less than or equal to 130, with preference for norbornene, 2,5-norbornadiene, 5-ethylidene-2-norbornene and 5-vinyl-2-norbornene.

The carboxylic acids involved are advantageously acetic acid, propionic acid, n-butyric acid, isobutyric acid, n-valerianic acid, isovalerianic acid, n-capronic acid, isocapronic acid or 2-methyl valerianic acid.

The aldehydes involved are advantageously acetaldehyde, propionaldehyde, n-butyraldehyde, isobutyraldehyde, n-valeraldehyde, isovaleraldehyde, n-capronaldehyde, isocapronaldehyde or 2-methylvaleraldehyde.

The phenols involved are advantageously substituted phenols with a total of one or two C₁-C₄-alkyl- and/or C₁-C₄-alkoxy groups. Preferred phenols are 3-methyl phenol, 2-ethyl phenol, 4-ethyl phenol, 2-isopropyl phenol, 2-tert.-butyl phenol, 2-tert.-butyl-4-methyl phenol, 2-methoxy phenol, 2-methoxy-4-methyl phenol and 2-methyl-5-isopropyl phenol. Particular preference is for C₁-C₄-monoalkylated phenols.

Advantageous anisoles are anisole, 2-methyl anisole, 4-allyl anisole or 4-methyl anisole.

The pyrazines involved are advantageously alkylated and/or acylated pyrazines. Examples of advantageous pyrazines are 2-methyl pyrazine, 2-ethyl pyrazine, 2,3-dimethyl pyrazine, 2,3-diethyl pyrazine, 2,6-dimethyl pyrazine, 2,3-methyl ethyl pyrazine, 5,2-methyl ethyl pyrazine, 2,3,5-trimethyl pyrazine, 3,5,2-dimethyl ethyl pyrazine, 3,6,2-dimethyl ethyl pyrazine, 5,2,3-methyl diethyl pyrazine, tetramethyl-pyrazine, 2,3-methyl acetyl pyrazine or 2-acetyl pyrazine. Preference is for pyrazines with a total of one to three, and special preference is for those with one or two C₁-C₄-alkyl- and/or C₁-C₄-acyl groups.

The acylated pyrazines are preferably monoacylated and, out of particular preference, have one acetyl or propionyl group, preference here being for monoacetylated pyrazines, in particular 2-acetyl pyrazine.

The compounds from group C) are contained in the mixtures in accordance with the invention typically at 1-40% by weight, advantageously 2-30% by weight, and preferably 3-15% by weight.

The weight ratio of components B) to components C) is advantageously in the range 3:1 to 1:3, preferably in the range 2:1 to 1:2 with particular preference for 1.2:1-1:1.2.

Common antioxidants can be added to the odorizers in accordance with the invention in order, for example, to improve stability. Examples of these include vitamin C and derivatives (e.g. ascorbylpalmitate, ascorbylacetate), tocopherol and derivatives (e.g. vitamin E, vitamin E—acetate), vitamin A and derivates (vitamin A—palmitate) phenol benzylamine, formic acid, acetic acid, benzoic acid, sorbic acid, hexamethylenetetramine, tert.-butylhydroxytoluol, tert.-butylhydroxyanisol, α-hydroxy acids (e.g. citric acid, lactic acid, malic acid), and hydroquinone monomethyl ether. Preferred antioxidants are tert.-butylhydroxytoluol (BHT, Jonol), tert.-butylhydroxyanisol und hydroquinone monomethyl ether.

The addition of antioxidants in particular gives the mixtures in accordance with the invention and also the odorised liquid gas greater storage stability. Storage stability tests have shown that the warning smell of the mixtures in accordance with the invention remains extensively unchanged over a period of 3 months at 20° C. or 14 days at 40° C. (incubator).

Several antioxidants can also be added to an odorizer. Advantageously the odorizers contain one, two or three antioxidants. One or two antioxidants are preferred.

The total quantity of antioxidants in the odorizer is normally in the range 0.05-2% by weight, preferably in the range 0.1-1% by weight, and out of particular preference in the range 0.3-0.8% by weight.

The quantity of odorizers in accordance with the invention with reference to the liquid gas to be odorized is typically in the range 5-100 mg/kg, preferably 5-50 mg/kg, out of particular preference 10-40 mg/kg and out of quite special preference 12-30 mg/kg.

The warning smell of the odorized liquid gas in accordance with the invention was clearly noticed by a group of testers even at a dilution of liquid gas in air in the range 1:200-1:2000.

The presence of the components C) in the mixtures in accordance with the invention meant that a better warning smell was achieved compared with mixtures that merely contained components A) and B).

Preferred mixtures in accordance with the invention contain
A) at least two different acrylic acid-C₁-C₄-alkylesters;
B) at least a compound from the group comprising C₁-C₈-mercaptans, C₄-C₈-thiophenes, C₂-C₈-sulphides or C₂-C₈-disulphides;
C) at least a compound from the group comprising norbornenes, C₂-C₅-carboxylic acids, C₂-C₅-aldehydes, C₆-C₁₀ phenols, C₇-C₁₀ anisoles or C₄-C₁₀ pyrazines;
D) at least an antioxidant

Particularly preferred mixtures in accordance with the invention contain
A) acrylic acid methyl esters, acrylic acid ethyl esters and if necessary a further acrylic acid —C₁-C₆-alkylester;
B) at least a compound from the group comprising thiopenes, tetrahydrothiophenes, dimethyl sulphides, diethyl sulphides, di-n-propyl sulphides, diisopropyl sulphides, dimethyl disulphides, diethyl disulphides, di-n-propyl disulphides, diisopropyl disulphides or mercaptans of formula (I) 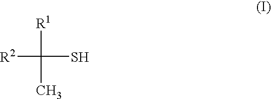
where
R¹ signifies hydrogen, methyl or ethyl, preferably methyl, and
R² signifies an alkyl group with between 1 and 4 carbon atoms, preferably methyl, ethyl, isopropyl, isobutyl or tert.-butyl;
C) at least a compound from the group comprising norbornenes, 2,5-norbonadienes, C₂-C₅-carboxylic acids, C₂-C₅-aldehydes, C₁-C₄-monoacetylated pyrazines, and C₁-C₄ monoalkylated phenols;
D) at least an antioxidant

Quite particularly preferred mixtures essentially comprise
A) acrylic acid methyl esters and acrylic acid ethyl esters;
B) tert.-butylmercaptans;
C) at least a compound from the group comprising norbornenes, propionaldehydes, isovaleraldehydes, isovalerianic acids, 2-ethyl phenols, 4-ethyl phenols, 2-acetyl pyrazines;
D) at least an antioxidant.

The following examples explain the invention:

Unless otherwise stated all data relate to the weight.

Me-Ac: methyl acrylate; Et-Ac: ethyl acrylate; Bu-Ac: n-butyl acrylate; DEP: diethyl phthalate; EtSH: ethyl mercaptan; iPrSH: isopropyl mercaptan, TBM: tert.-butyl mercaptan, THM: tert.-hexyl mercaptan; THT: tetrahydrothiophene, DES: diethyl sulphide, Norb: norbornene, Prop: propionaldehyde, IsoVS: isovalerianic acid, Iso VA: isovalerianic aldehyde, 2-EtPh: 2-ethyl phenol; 4-EtPh: 4-ethyl phenol, AcPyr: 2-acetyl pyrazine, BHT: tert.-butylhydroxytoluol; BHA: tert.-butylhydroxyanisol.

### EXAMPLE 1

The odorizers underwent an olfactory assessment in concentrations of 10, 25 and 50 mg/kg of liquid gas (20.3% propane, 78.6% butane, 1.1% other hydrocarbons) of their warning smell and their warning intensity compared with liquid gas that contained the same concentrations of ethyl mercaptan or the odourless diethyl phthalate (blank value). These concentrations correspond to the typical concentrations of odorizers in the liquid gas.

The assessment was on a scale of 0-10, where 0 meant odourless, 1 very weak/little warning, and 10 very strong warning. The stated values are averages. The industry standard ethyl mercaptan was given a value of 10 and diethyl phthalate a value of 0.

An aerosol was filled with liquid gas with the abovementioned composition and a corresponding quantity of odorizer. After a rest phase of approximately 24 hours the odorized liquid gases underwent olfactory assessment. The test was performed at room temperature (approximately 20° C.) in such a way that the gas phase of the odorised liquid gas was sprayed from the aerosol can into a smelling beaker and the gas area in this smelling beaker underwent an olfactory assessment from a group of trained testers (8 to 12 people).

The results for the three concentrations analysed (10, 25 and 50 mg/kg of liquid gas) were extensively the same. The assessments indicated in the following were determined for a concentration of 35 mg/kg liquid gas.

Table 1 gives a comparison between references and components of the mixtures in accordance with the invention.

TABLE 1

| | | Me— | Et— | | |
|---|---|---|---|---|---|
| Substance | Substance | Ac | Ac | DEP | Assessment |
| Ethyl mercaptan | 100 | — | — | — | 10 |
| Diethyl phthalate | | — | — | 100 | 0 |
| Acrylic acid ethyl ester | | — | 100 | — | 5 |
| Acrylic acid methyl | | 100 | — | — | 4.5 |
| ester | | | | | |
| Acrylic acid-n-butyl | 100 | — | — | — | 3.5 |
| ester | | | | | |
| Tert.-butyl mercaptan | 25 | — | — | 75 | 7 |
| Propionaldehyde | 100 | — | — | — | 3 |
| Norbornene | 100 | — | — | — | 3 |
| Isovalerianic acid | 100 | — | — | — | 3.5 |
| Isovaleraldehyde | 100 | — | — | — | 3.5 |
| 2-Ethyl phenol | 100 | — | — | — | 3 |
| 4-Ethyl phenol | 100 | — | — | — | 3 |
| 2-Acetyl pyrazine | 100 | — | — | — | 3.5 |

### EXAMPLE 2

Table 2 gives the assessments for propionaldehyde (Prop) as component C). These were performed as described in Example 1.

TABLE 2

| Et—Ac | Me—Ac | TBM | Prop | DEP | Assessment |
|---|---|---|---|---|---|
| — | — | 5 | — | 95 | 6.5 |
| 58 | 36 | 5 | — | 1 | 7 |
| 58 | 36 | 5 | 1 | — | 8 |
| 56 | 34 | 5 | — | 5 | 7.5 |
| 56 | 34 | 5 | 5 | — | 9.5 |
| 53 | 32 | 5 | — | 10 | 8 |
| 53 | 32 | 5 | 10 | — | 9 |
| — | — | 10 | — | 90 | 6.5 |
| 53 | 32 | 10 | — | 5 | 7.5 |
| 53 | 32 | 10 | 5 | — | 8.5 |
| 50 | 30 | 10 | — | 10 | 8 |
| 50 | 30 | 10 | 10 | — | 9.5 |
| — | — | 20 | — | 80 | 7 |
| 48 | 29 | 20 | — | 3 | 8.5 |
| 48 | 29 | 20 | 3 | — | 9 |
| 43 | 27 | 20 | — | 10 | 9 |
| 43 | 27 | 20 | 10 | — | 9.5 |
| 40 | 25 | 20 | — | 15 | 9.5 |
| 40 | 25 | 20 | 15 | — | 10 |

### EXAMPLE 3

Table 3 gives the assessments for norbornene (Norb) as component C). These were ed as described in Example 1.

TABLE 3

| Et—Ac | Me—Ac | TBM | Norb | DEP | Assessment |
|---|---|---|---|---|---|
| 56 | 34 | 5 | — | 5 | 7.5 |
| 56 | 34 | 5 | 5 | — | 9 |
| 53 | 32 | 5 | — | 10 | 7.5 |
| 53 | 32 | 5 | 10 | — | 8.5 |
| 53 | 32 | 10 | — | 5 | 7.5 |
| 53 | 32 | 10 | 5 | — | 9 |
| 50 | 30 | 10 | — | 10 | 8 |
| 50 | 30 | 10 | 10 | — | 9.5 |
| 48 | 29 | 20 | — | 3 | 8.5 |
| 48 | 29 | 20 | 3 | — | 9 |
| 37 | 23 | 20 | — | 20 | 9.5 |
| 37 | 23 | 20 | 20 | — | 10 |

### EXAMPLE 4

Table 4 gives the assessments for isovalerianic acid (IsoVS) as component C). These formed as described in Example 1.

TABLE 4

| Et—Ac | Me—Ac | TBM | IsoVS | DEP | Assessment |
|---|---|---|---|---|---|
| 57 | 35 | 5 | — | 3 | 7.5 |
| 57 | 35 | 5 | 3 | — | 8.5 |
| 56 | 34 | 5 | — | 5 | 7.5 |
| 56 | 34 | 5 | 5 | — | 9.5 |
| 50 | 30 | 5 | — | 15 | 8 |
| 50 | 30 | 5 | 15 | — | 9 |
| 53 | 32 | 10 | — | 5 | 7.5 |
| 53 | 32 | 10 | 5 | — | 9.5 |
| 50 | 30 | 10 | — | 10 | 8 |
| 50 | 30 | 10 | 10 | — | 10 |
| 46 | 29 | 10 | — | 15 | 8 |
| 46 | 29 | 10 | 15 | — | 9.5 |
| 48 | 29 | 20 | — | 3 | 8.5 |
| 48 | 29 | 20 | 3 | — | 9 |
| 46 | 29 | 20 | — | 7.5 | 9 |
| 46 | 29 | 20 | 7.5 | — | 9.5 |
| 40 | 25 | 20 | — | 15 | 9.5 |
| 40 | 25 | 20 | 15 | — | 10 |

### EXAMPLE 5

Table 5 gives the assessments for 2-ethyl phenol (2-EthPh) as component C). These formed as described in Example 1.

TABLE 5

| Et—Ac | Me—Ac | TBM | 2-EtPh | DEP | Assessment |
|---|---|---|---|---|---|
| 56 | 34 | 5 | — | 5 | 7.5 |
| 56 | 34 | 5 | 5 | — | 8.5 |
| 53 | 32 | 5 | — | 10 | 7.5 |
| 53 | 32 | 5 | 10 | — | 8.0 |
| 53 | 32 | 10 | — | 5 | 7.5 |
| 53 | 32 | 10 | 5 | — | 8.5 |
| 50 | 30 | 10 | — | 10 | 8 |
| 50 | 30 | 10 | 10 | — | 9.0 |
| 48 | 29 | 20 | — | 3 | 8.5 |
| 48 | 29 | 20 | 3 | — | 9 |
| 40 | 25 | 20 | — | 15 | 9.5 |
| 40 | 25 | 20 | 15 | — | 9.5 |

### EXAMPLE 6

Table 6 gives the assessments for 4-ethyl phenol (4-EthPh) as component C). These formed as described in Example 1.

TABLE 6

| Et—Ac | Me—Ac | TBM | 4-EtPh | DEP | Assessment |
|---|---|---|---|---|---|
| 56 | 34 | 5 | — | 5 | 7.5 |
| 56 | 34 | 5 | 5 | — | 8.5 |
| 53 | 32 | 5 | — | 10 | 7.5 |
| 53 | 32 | 5 | 10 | — | 8.0 |
| 53 | 32 | 10 | — | 5 | 7.5 |
| 53 | 32 | 10 | 5 | — | 8.5 |
| 50 | 30 | 10 | — | 10 | 8 |
| 50 | 30 | 10 | 10 | — | 9.0 |
| 48 | 29 | 20 | — | 3 | 8.5 |
| 48 | 29 | 20 | 3 | — | 9 |
| 40 | 25 | 20 | — | 15 | 9.5 |
| 40 | 25 | 20 | 15 | — | 9.5 |

### EXAMPLE 7

Table 7 gives the assessments for 2-acetyl pyrazine (AcPyr) as component C). These formed as described in Example 1.

TABLE 7

| Et—Ac | Me—Ac | TBM | AcPyr | DEP | Assessment |
|---|---|---|---|---|---|
| 56 | 34 | 5 | — | 5 | 7.5 |
| 56 | 34 | 5 | 5 | — | 8. |
| 53 | 32 | 5 | — | 10 | 7.5 |
| 53 | 32 | 5 | 10 | — | 8.5 |
| 53 | 32 | 10 | — | 5 | 7.5 |
| 53 | 32 | 10 | 5 | — | 8.5 |
| 50 | 30 | 10 | — | 10 | 8 |
| 50 | 30 | 10 | 10 | — | 8 |
| 48 | 29 | 20 | — | 3 | 8.5 |
| 48 | 29 | 20 | 3 | — | 9 |
| 40 | 25 | 20 | — | 15 | 9.5 |
| 40 | 25 | 20 | 15 | — | 8.5 |

### EXAMPLE 8

Table 8 gives the assessments for isovaleraldehyde (IsoVA) as component C). These formed as described in Example 1.

TABLE 8

| Et—Ac | Me—Ac | TBM | IsoVA | DEP | Assessment |
|---|---|---|---|---|---|
| 57 | 35 | 5 | — | 3 | 7.5 |
| 57 | 35 | 5 | 3 | — | 8.5 |
| 56 | 34 | 5 | — | 5 | 7.5 |
| 56 | 34 | 5 | 5 | — | 9.5 |
| 50 | 30 | 5 | — | 15 | 8 |
| 50 | 30 | 5 | 15 | — | 8.5 |
| 53 | 32 | 10 | — | 5 | 7.5 |
| 53 | 32 | 10 | 5 | — | 9.5 |
| 50 | 30 | 10 | — | 10 | 8 |
| 50 | 30 | 10 | 10 | — | 9.5 |
| 46 | 29 | 10 | — | 15 | 8 |
| 46 | 29 | 10 | 15 | — | 9.5 |
| 48 | 29 | 20 | — | 3 | 8.5 |
| 48 | 29 | 20 | 3 | — | 9 |
| 46 | 29 | 20 | — | 7.5 | 9 |
| 46 | 29 | 20 | 7.5 | — | 9.5 |
| 40 | 25 | 20 | — | 15 | 9.5 |
| 40 | 25 | 20 | 15 | — | 10 |

### EXAMPLE 9

Table 9 shows odorizers in accordance with the invention each with a component C). The proportion of component C) in the odorizer was 5% and the mixtures contained 0.5% BHT as an antioxidant.

TABLE 9

| | Et— | Me— | Bu— | | | | |
|---|---|---|---|---|---|---|---|
| C) - 5% | Ac | Ac | Ac | TBM | THM | iPrSH | EtSH |
| Norbornene | 49.5 | 30 | 10 | 5 | — | — | — |
| Norbornene | 50.5 | 34 | — | 5 | 2 | 3 | — |
| 2,5-Norbornadiene | 49.5 | 36 | 5 | 3 | — | — | 1 |
| 2,5-Norbornadiene | 38 | 35 | 15 | 4.5 | — | 2 | — |
| Propionaldehyde | 35 | 29.5 | 23 | 5 | 2 | — | — |
| Propionaldehyde | 28 | 50.5 | 9 | 3 | 1 | 2 | 1 |
| Isobutyraldehyde | 35 | 35 | 20 | 2.5 | — | — | 2 |
| n-Valeraldehyde | 55 | 36.5 | — | — | — | — | 3 |
| n-Butyric acid | 48.5 | 34 | 8 | 2 | 2 | — | — |
| Isovalerianic acid | 44.5 | 36 | 10 | 4 | — | — | — |
| Isovalerianic acid | 59.5 | 29 | — | 2 | 1 | 2 | 1 |
| 2-Ethyl phenol | 47.5 | 34 | 8 | 5 | — | — | — |
| 2-tert.-Butyl phenol | 50 | 41.5 | — | — | — | 3 | — |
| 2-Methoxyphenol | 49.5 | 40 | — | 3 | 2 | — | — |
| 4-Allyl anisole | 40 | 40 | 9.5 | 5 | — | — | — |
| 2-Methyl anisole | 50 | 30 | 10 | 2.5 | 2 | — | — |
| Isovaleraldehyde | 55 | 36.5 | — | 3 | — | — | — |
| Isovaleraldehyde | 55 | 36.5 | — | — | — | — | 3 |
| 4-Ethyl phenol | 47.5 | 34 | 8 | 5 | — | — | — |
| 4-Ethyl phenol | 55 | 36.5 | — | 3 | — | — | — |
| 2,3-Methyl ethyl | 55 | 36.5 | — | 3 | — | — | — |
| pyrazine | | | | | | | |
| 2-Acetyl pyrazine | 50 | 30 | 10 | 2.5 | 2 | — | — |
| 2-Acetyl pyrazine | 59.5 | 30 | — | 2 | 1 | 1 | 1 |

### EXAMPLE 10

Table 10 shows odorizers in accordance with the invention containing ethyl acrylate, methyl acrylate, at least a component C) from the group comprising Prop, Norb, IsoVS, 2-EtPh and at least a further component C), as well as an antioxidant. The odorizers contained of components A) 50% ethyl acrylate and 36% methyl acrylate, and of components C from column one 3.3%. The mixtures contained 0.7% hydroquinone monomethyl ether as antioxidant.

TABLE 10

| | | | | 2- | | | |
|---|---|---|---|---|---|---|---|
| C) - 3.3% | Prop | Norb | IsoVS | EtPh | TBM | THM | iPrSH |
| Norbornene | 5 | — | — | — | 5 | — | — |
| Norbornene | — | — | — | 4 | 3 | — | 3 |
| Norbornene | — | — | 3 | 1 | 4 | 2 | — |
| 2,5- | — | 4.5 | — | — | 4.5 | 1 | — |
| Norbornadiene | | | | | | | |
| 2,5- | 3 | 2 | — | — | 5 | — | — |
| Norbornadiene | | | | | | | |
| Propionaldehyde | — | — | — | 4 | 3 | — | 2 |
| n-Valeraldehyde | 3 | 1 | — | — | 4 | 2 | — |
| n-Butyric acid | — | 2 | 2 | 1 | 5 | — | — |
| Isovalerianic acid | — | 3 | — | 2 | 5 | — | — |
| 2-Ethyl phenol | 2 | 3 | — | — | 2 | 3 | — |
| 2-tert.-Butyl | — | 2 | — | 2.5 | 5.5 | — | — |
| phenol | | | | | | | |
| 2-Methoxyphenol | 5 | 1 | — | — | — | — | 4 |
| 4-Allyl anisole | — | 2 | — | 2 | 4 | 2 | — |
| 2-Methyl anisole | — | — | 4 | 1 | 5 | — | — |
| Isovaleraldehyde | 5 | — | — | — | — | 5 | — |
| Isovaleraldehyde | 3 | 2 | — | — | 2 | — | 3 |
| 4-Ethyl phenol | — | — | 3 | 1 | 1 | 5 | — |
| 4-Ethyl phenol | 2 | — | — | 4 | 4 | — | — |
| 2,3-Methyl | 3 | 2 | — | — | 2 | — | 3 |
| ethylpyrazine | | | | | | | |
| 2,6-Dimethyl | — | 5 | — | — | 5 | — | — |
| pyrazine | | | | | | | |
| 2-Acetyl pyrazine | 3 | — | — | 2 | 3 | 1 | 1 |
| 2-Acetyl pyrazine | — | 5 | — | — | 5 | — | — |

### EXAMPLE 11

Table 11 shows odorizers in accordance with the invention containing ethyl acrylate, methyl acrylate, at least a component C) from the group comprising AcPyr, IsoVA, 4-EtPh and at least a further component C), as well as an antioxidant. The odorizers contained of components A) 50% ethyl acrylate and 36% methyl acrylate, and of components C from column one 3.3%. The mixtures contained 0.7% BHA as antioxidant.

TABLE 11

| C) - 3.3% | AcPyr | IsoVA | 4-EtPh | THT | TBM | iPrSH | DES |
|---|---|---|---|---|---|---|---|
| Norbornene | 5 | — | — | 5 | — | — | — |
| Norbornene | — | — | 4 | — | 3 | 3 | — |
| Norbornene | — | 3 | 1 | 2 | 4 | — | — |
| 2,5-Norbornadiene | 4.5 | — | — | — | 4.5 | — | 1 |
| 2,5-Norbornadiene | 3 | — | — | 2 | 5 | — | — |
| Propionaldehyde | — | — | 5 | — | 3 | 2 | — |
| n-Valeraldehyde | — | 4 | — | 2 | 4 | — | — |
| n-Butyric acid | — | 2 | 1 | 5 | — | — | 2 |
| Isovalerianic acid | 3 | — | 2 | — | 5 | — | — |
| 2-Ethyl phenol | 2 | 3 | — | — | 2 | — | 3 |
| 2-tert.-Butyl phenol | 3 | — | 2 | — | 5 | — | — |
| 2-Methoxyphenol | 5 | — | — | 1 | — | 4 | — |
| 4-Allyl anisole | — | 2 | 2 | — | 4 | — | 2 |
| 2-Methyl anisole | — | 4 | 1 | — | 5 | — | — |
| Isovaleraldehyde | 3 | — | — | 5 | — | — | 2 |
| Isovaleraldehyde | — | — | 5 | 3 | 2 | — | — |
| 4-Ethyl phenol | 5 | — | — | 5 | — | — | — |
| 4-Ethyl phenol | 2 | 3 | — | — | 3 | — | 2 |
| 2,3-Methyl ethylpyrazine | — | 2 | 2 | — | 4 | — | 2 |
| 2,3,5-Trimethyl | 5 | — | — | 5 | — | — | — |
| pyrazine | | | | | | | |
| 2-Acetyl pyrazine | — | 2.5 | 2.5 | 2.5 | — | 2.5 | — |
| 2-Acetyl pyrazine | — | 2 | 1 | 3 | 3 | 1 | — |

### EXAMPLE 12

In order to check the thermal stability the odorized liquid gases underwent olfactory assessment as described in Example 1 first after 24 hours at 20° C. (fresh) and after 14 days storage at 40° C. (storage). The quantities of odorizer added to the liquid gases was 35 mg/kg. Table 12 gives the results of the comparison between ethyl mercaptan and an mixture A in accordance with the invention comprising 34% Me-Ac, 55.5% Et-Ac, 5% TBM, 5% Prop and 0.5% BHT.

TABLE 12

| | | | Assessment | Assessment |
|---|---|---|---|---|
| Substance(s) | Propane | n-Butane | Fresh | Storage |
| EtSH | 30 | 70 | 10 | 5 |
| EtSH | 70 | 30 | 10 | 4 |
| Mixture A | 30 | 70 | 9.5 | 9.5 |
| Mixture A | 70 | 30 | 9.5 | 9 |

Following storage the odorized liquid gas with EsTH had changed significantly. The smell no longer produced any association with danger, since it was reminiscent of cooked cabbage.

Even after a period of storage of 6 months at 20° C. the warning smell of mixture A in the propane/n-butane mixtures was still exceedingly noticeable.

In the propane/n-butane mixtures with EtSH added after storage at 20° C. for a few weeks a definite smell of cabbage could be noticed also.

1. Mixtures containing
A) at least two different acrylic acid-C₁-C₆-alkylesters;
B) at least a compound from the group comprising C₁-C₈-mercaptans, C₄-C₁₂-thiophenes, C₂-C₈-sulphides or C₂-C₈-disulphides;
C) at least a compound from the group comprising norbornenes, C₁-C₅-carboxylic acids, C₁-C₈-aldehydes, C₆-C₁₄ phenols, C₇-C₁₄ anisoles or C₄-C₁₄ pyrazines;
D) if necessary an antioxidant.

2. Mixtures according to claim 1, containing:
A) at least two different acrylic acid-C₁-C₄-alkylesters;
B) at least a compound from the group comprising C₁-C8-mercaptans, C₄-C₈-thiophenes, C₂-C₈-sulphides or C₂-C₈-disulphides;
C) at least a compound from the group comprising norbornenes, C₂-C₅-carboxylic acids, C₂-C₅-aldehydes, C₆-C₁₀ phenols, C₇-C₁₀ anisoles or C₄-C₁₀ pyrazines;
D) at least an antioxidant.

3. Mixtures according to claim 1, containing:
A) acrylic acid methyl esters, acrylic acid ethyl esters and if necessary a further acrylic acid-C₁-C₆-alkylester;
B) at least a compound from the group comprising thiopenes, tetrahydrothiophenes, dimethyl sulphides, diethyl sulphides, di-n-propyl sulphides, diisopropyl sulphides, dimethyl disulphides, diethyl disulphides, di-n-propyl disulphides, diisopropyl disulphides or mercaptans of formula (I) 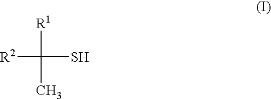
where
R¹ signifies hydrogen, methyl or ethyl, preferably methyl, and
R² signifies an alkyl group with between 1 and 4 carbon atoms, preferably methyl, ethyl, isopropyl, isobutyl or tert.-butyl;
C) at least a compound from the group comprising norbornene, 2,5-norbonadienes, C₂-C₅-carboxylic acids, C₂-C₅-aldehydes, C₁-C₄-monoacetylated pyrazines, and C₁-C₄ monoalkylated phenols;
D) at least an antioxidant.

4. Mixtures according to claim 1, containing:
A) acrylic acid methyl esters and acrylic acid ethyl esters;
B) tert.-butylmercaptans;
C) at least a compound from the group comprising norbornenes, propionaldehydes, isovaleraldehydes, isovalerianic acids, 2-ethyl phenols, 4-ethyl phenols, 2-acetyl pyrazines;
D) at least an antioxidant.

5. Mixtures according to claim 1, wherein said mixtures contain an antioxidant chosen from among tert.-butyl hydroxytoluol, tert.-butyl hydroxyanisole or hydroquinone mono-methylether.

6. Mixtures according to claim 1, containing:
60-97% by weight of components A),
1-40% by weight of components B),
1-40% by weight of components C)
and 0.05-2% by weight of components D).

7. Mixtures according to claim 6, containing:
70-95% by weight of components A),
2-30% by weight of components B),
2-30% by weight of components C)
and 0.1-1% by weight of components D).

8. Mixtures according to claim 1, having a weight ratio between components B) and components C) in the range of 3:1 to 1:3.

9. (canceled)

10. Liquid gas containing mixtures according to claim 1.

11. Method for providing a noticeable odor to virtually odorless liquid gas, comprising: adding a mixture according to claim 1 to the liquid gas.

12. Method according to claim 11, characterised in that the mixture is added to the liquid gas in a quantity of 5-100 mg/kg of liquid gas.
